(19) United States
(12) Patent Application Publication
(10) Pub. No.: US 2021/0020318 A1
(43) Pub. Date: Jan. 21, 2021

(54) SYSTEMS AND METHODS FOR MEDICAL SESSION COMMUNICATION

(71) Applicant: Safa Movassaghi, Mission Viejo, CA (US)

(72) Inventor: Safa Movassaghi, Mission Viejo, CA (US)

(21) Appl. No.: 16/929,783

(22) Filed: Jul. 15, 2020

Related U.S. Application Data
(60) Provisional application No. 62/874,314, filed on Jul. 15, 2019.

(51) Int. Cl. G16H 80/00; G16H 10/60; G16H 10/20; G16H 15/00; G16H 50/20; G06Q 40/02; G06Q 20/12; G16H 40/67; H04L 29/06; G06N 20/00; G06F 40/40

(52) U.S. Cl. CPC G16H 80/00; G16H 10/60; G16H 10/20; G16H 15/00; G16H 50/20; G06F 40/40; G06Q 20/127; G16H 40/67; H04L 63/0428; G06N 20/00; H04L 65/1066; G06Q 40/02

(57) ABSTRACT

Disclosed herein are methods and systems of providing computerized medical communications. An exemplary method includes receiving patient account data associated with a patient and generating a plurality of questions configured to elicit responses from the patient. The method transmits the generated plurality of questions to the patient and subsequently receives response data from the patient wherein the response data is generated based on the transmitted plurality of questions. Virtual chart data is generated with a virtual chart logic based on the received response data. Provider data comprising subscribed providers is received and a secure session is generated between the patient and a provider based on at least the patient account data and the provider data. The virtual chart data is provided to the medical care provider during the session; and, upon termination, a method of acceptance is provided to the patient to indicate an approval of the secured session termination.

### PRIORITY

This application claims the benefit of and priority to U.S. Provisional Application No. 62/874,314, filed on Jul. 15, 2019, the entirety of said application being incorporated herein by reference.

### FIELD

The field of the present disclosure generally relates to medical care. More particularly, the field of the invention relates to improving patient care through the creation of virtual sessions, patient charts, and machine-learning generated session templates for providers.

### BACKGROUND

The healthcare industry is growing constantly every year. This continued growth creates new challenges for patients seeking treatment and for medical providers attempting to deliver sufficient care to as many patients as possible. Insurance costs continue to rise, affecting healthcare affordability for many younger patients as well as those with limited means. Additionally, most, if not all urgent care centers only provide services until the evening hours of seven to nine o'clock.

Patients seeking healthcare may not have access to other medical providers after these hours unless they visit a hospital emergency room, leading to increased costs, overcrowding, and disease spreading. Not only is the cost to provide healthcare services increased in an emergency room setting, but the costs for the patient are often higher as well. This increased patient-end costs typically come in the form of higher deductibles. Emergency rooms are also not the optimal setting for families with special needs or small children. Patients may further put off needed visits for fear of exposing themselves to other contagious illnesses. Often, the chief complaint of a patient presenting in the emergency room is simple or minor in nature and may be handled in a non-emergency setting such as an urgent care facility. As such, a solution to provide easier and more available access to non-emergency medical care is needed.

### BRIEF DESCRIPTION OF THE DRAWINGS

The drawings refer to embodiments of the present disclosure in which:

FIG. 1 depicts an exemplary system diagram of a medical session communication system in accordance with various embodiments of the invention;

FIG. 2 depicts an exemplary block diagram of a memory within medical session communication system in accordance with an embodiment of the invention;

FIG. 3 depicts an exemplary flowchart of a medical session communication process in accordance with an embodiment of the invention;

FIG. 4 depicts an exemplary flowchart of a pre-session account setup process in accordance with an embodiment of the invention;

FIG. 5 depicts an exemplary flowchart of session template generation in accordance with an embodiment of the invention; and

FIG. 6 depicts an exemplary flowchart of a secure session interaction in accordance with an embodiment of the invention.

While the present disclosure is subject to various modifications and alternative forms, specific embodiments thereof have been shown by way of example in the drawings and will herein be described in detail. The invention should be understood to not be limited to the particular forms disclosed, but on the contrary, the intention is to cover all modifications, equivalents, and alternatives falling within the spirit and scope of the present disclosure.

### DETAILED DESCRIPTION

In the following description, numerous specific details are set forth in order to provide a thorough understanding of the present disclosure. It will be apparent, however, to one of ordinary skill in the art that the invention disclosed herein may be practiced without these specific details. In other instances, specific numeric references such as “first device,” may be made. However, the specific numeric reference should not be interpreted as a literal sequential order but rather interpreted that the “first device” is different than a “second device.” Thus, the specific details set forth are merely exemplary. The specific details may be varied from and still be contemplated to be within the spirit and scope of the present disclosure.

Aspects of the present disclosure may be embodied as an apparatus, system, method, or computer program product. Accordingly, aspects of the present disclosure may take the form of an entirely hardware embodiment, an entirely software embodiment (including firmware, resident software, micro-code, or the like) or an embodiment combining software and hardware aspects that may all generally be referred to herein as a “function,” “module,” “apparatus,” or “system.” Furthermore, aspects of the present disclosure may take the form of a computer program product embodied in one or more non-transitory computer-readable storage media storing computer-readable and/or executable program code. Many of the functional units described in this specification have been labeled as logic, in order to emphasize their implementation independence more particularly. For example, a logic may be implemented as a hardware circuit comprising custom VLSI circuits or gate arrays, off-the-shelf semiconductors such as logic chips, transistors, or other discrete components. A logic may also be implemented in programmable hardware devices such as via field programmable gate arrays, programmable array logic, programmable logic devices, or the like.

Logics may also be implemented at least partially in software for execution by various types of processors. An identified logic comprising executable code may, for instance, comprise one or more physical or logical blocks of computer instructions that may, for instance, be organized as an object, procedure, or function. Nevertheless, the executables of an identified logic need not be physically located together but may comprise disparate instructions stored in different locations which, when joined logically together, comprise the logic and achieve the stated purpose for the logic.

Indeed, a logic comprising executable code may include a single instruction, or many instructions, and may even be distributed over several different code segments, among different programs, across several storage devices, or the like. Where a logic or portions of a logic are implemented in software, the software portions may be stored on one or more computer-readable and/or executable storage media. Any combination of one or more computer-readable storage media may be utilized. A computer-readable storage medium may include, for example, but not limited to, an electronic, magnetic, optical, electromagnetic, infrared, or semiconductor system, apparatus, or device, or any suitable combination of the foregoing, but would not include propagating signals. In the context of this document, a computer readable and/or executable storage medium may be any tangible and/or non-transitory medium that may contain or store a program for use by or in connection with an instruction execution system, apparatus, processor, or device.

Computer program code for carrying out operations for aspects of the present disclosure may be written in any combination of one or more programming languages, including an object-oriented programming language such as Python, Java, Smalltalk, C++, C#, Objective C, or the like, conventional procedural programming languages, such as the “C” programming language, scripting programming languages, and/or other similar programming languages. The program code may execute partly or entirely on one or more of a user's computer and/or on a remote computer or server over a data network or the like.

The term “cloud-based” generally refers to a hosted service that is remotely located from a data source and configured to receive, store and process data delivered by the data source over a network, including a self-hosted and third-party hosted service. Cloud-based systems may be configured to operate as a public cloud-based service, a private cloud-based service or a hybrid cloud-based service. A “public cloud-based service” may include a third-party provider that supplies one or more servers to host multi-tenant services. Examples of a public cloud-based service include Amazon Web Services® (AWS®), Microsoft® Azure™, and Google® Compute Engine™ as examples. In contrast, a “private” cloud-based service may include one or more servers that host services provided to a single subscriber (enterprise) and a hybrid cloud-based service may be a combination of both a public cloud-based service and a private cloud-based service.

The term “client device” or “server” should be generally construed as electronics with data processing capability and/or a capability of connecting to any type of network, such as a public network (e.g., Internet), a private network (e.g., a wireless data telecommunication network, a local area network “LAN”, etc.), or a combination of networks. Examples of a client devices/servers may include, but are not limited or restricted to, the following: a stand-alone electronic device, a router; an info-entertainment device, vehicles, or other personal device (e.g., a laptop, a smartphone, a tablet, a desktop computer, a netbook, gaming console, a medical device, or any general-purpose or special-purpose, user-controlled electronic device).

The term “coupled” is defined as meaning connected either directly to the component or indirectly to the component through another component. Further, as used herein, the terms “about,” “approximately,” or “substantially” for any numerical values or ranges indicate a suitable dimensional tolerance that allows the part or collection of components to function for its intended purpose as described herein.

Reference throughout this specification to “one embodiment,” “an embodiment,” or similar language means that a particular feature, structure, or characteristic described in connection with the embodiment is included in at least one embodiment of the present disclosure. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to”, unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Further, as used herein, reference to reading, writing, storing, buffering, and/or transferring data can include the entirety of the data, a portion of the data, a set of the data, and/or a subset of the data. Likewise, reference to reading, writing, storing, buffering, and/or transferring non-host data can include the entirety of the non-host data, a portion of the non-host data, a set of the non-host data, and/or a subset of the non-host data.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps, or acts are in some way inherently mutually exclusive.

In response to the problems described above, the present disclosure describes a medical session communication system. One or more secure medical-related sessions can be created that bring together one or more patients with one or more doctors or medical providers. In order to facilitate these sessions, direct payment may be made by the patient to a “balance” within the patient's account within the system. The balance may be in the form of stored cash value or a number of fixed block credits. When the patient wants to talk with a doctor about a particular symptom, issue, and/or problem, a request to start a session may be made once the patient has successfully set up an account.

Often, the medical sessions created by the system involve teleconferenced or other remote communications between patients and medical providers. In this way, patients in under-served demographics may have easier access to medical advice and care. The medical session communication system can further generate an account and virtual chart for each patient that can then be utilized by the medical provider during sessions. Medical sessions can often be a discussion, or when appropriate, an audio/visual conference that may allow a medical professional to see injuries or other physical indicators necessary to provide a reasonable standard of care to the patient.

In order to facilitate these discussions, the present invention may utilize one or more machine learning systems to generate a session template that can guide the session and give the medical provider pre-configured questions to answer and tests to conduct based on the input data given by the patient (which may include virtual chart data, symptom/issue data, etc.). The session template can help reduce errors as well as verify (and avoid liability of) a medical provider's standard of care. In some cases, the session template must be completed successfully (which may be defined on a symptom/issue by symptom/issue basis) in order to facilitate payment to the medical provider.

These medical sessions can provide ease of access and can also be configured as a subscription service, thereby allowing patients with lower financial means access to medical professionals. Additionally, because of the remote nature of the medical sessions, medical experts from distant locations can be utilized in a more efficient manner to examine and diagnose cases suitable to their training. This can allow connections between patients and medical providers internationally, assuming both patient and medical provider abide by local, state, and federal laws and regulations. The medical session communication system can determine the location of both the patient and medical professional and dynamically adjust any data and/or communication practices to comport with the current legal standards. As can be understood by those skilled in the art, the medical session communication system can provide interfaces and translation services for patients and/or medical providers in their native language dependent on their location or personal settings.

Medical sessions can occur in conjunction with patient data being gathered in real-time or near real-time by a plurality of medical devices that can be in communication with the system. By way of example and not limitation, a blood pressure device may be utilized to provide blood pressure data to the medical professional, or oxygen measurements are transmitted in real-time from a device connected to the medical session communication system application via Bluetooth or other data connection.

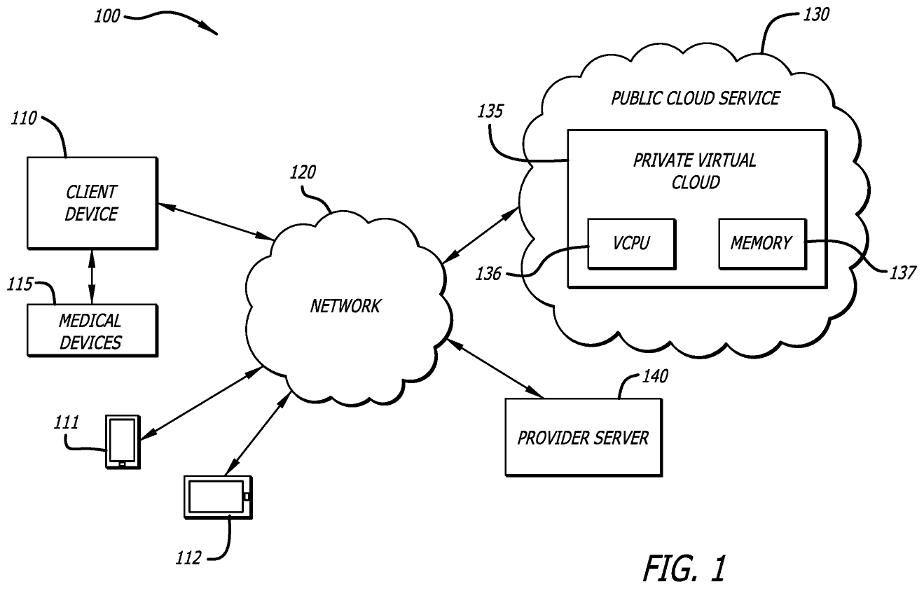

Referring to FIG. 1, an exemplary system diagram of a medical session communication system 100 in accordance with various embodiments of the invention is shown. The medical session communication system 100 may exist in a number of configurations. By way of example and not limitation, the medical session communication system 100 may be implemented through the use of a cloud-based service. FIG. 1 depicts a public cloud service 130 being accessible to a client device 110 through a connection to a network 120. In many embodiments, the network 120 is the Internet.

The client device 110 can be accessible to a patient seeking medical care. In certain embodiments, the client device 110 may be a personal computer, dedicated kiosk, or other tele-conferencing hardware and/or software. In a number of embodiments, the patient may have a mobile computing device such as a cellular phone 111 or tablet 112 which are also connected to the network 120 and may act as client devices similar to client device 110. It is contemplated that the mobile computing devices may comprise software (i.e. applications) that may communicate with the medical session communication system 100 without the need for additional hardware. Additionally, certain features of the cellular phone 111 or tablet 112 may be utilized to facilitate operation of the medical session communication system 100, including, but not limited to, microphones, cameras, text input, personal data, health-related data, payment data and/or biometric security/verification.

A portion of the potential patient pool for the medical session communication system 100 may use one or more medical devices 115, which may be commutatively coupled to the client device 110. It would be appreciated by those skilled in the art that certain medical devices 115 may be configured to provide data in a format suitable for transmission to a medical session or virtual chart associated with the patient owner of the medical devices 115. By way of example and not limitation, medical devices 115 may comprise dialysis machines, nebulizers, glucose meters, pulse oximeters, pacemakers, continuous positive airway pressure (CPAP) machines, and/or infusion pumps. As can be appreciated, nearly any medical device that can generate and transmit digital data may be utilized as a medical device 115 in the medical session communication system 100. Once on communication with the client device 110, the medical devices 115 may be configured to communication their data over the network 120 either directly or via a connection port utilized by the client device 110. In a variety of embodiments, the medical devices 115 may be commutatively coupled to the cellular phone 111 or tablet 112 instead of a client device 110. In other embodiments, the medical devices 115 may be configured to connect to the network 120 directly without the need of a connection to either a client device 110, or mobile computing device such as a cellular phone 111, or tablet 112.

The medical session communication system 100 depicted in FIG. 1 is implemented within a public cloud service 130, which itself may host a private virtual cloud 135. In many instances, a private virtual cloud 135 may be provisioned from a pool of available processing resources within the public cloud service 130. The private virtual cloud 135 and associated resources can be generated as part of an Infrastructure-as-a-Service (IaaS) model and comprise at least one instance of a vCPU 136 and a memory 137 communicatively coupled to the vCPU 136. It would be understood by those skilled in the art that the private virtual cloud 135 may comprise a variable number of vCPUs and memory stores as needed based on various factors including, but not limited to, the current available computing resources available on the system, or the current computational demands placed upon the private virtual cloud 135.

In many embodiments, the memory 137 may comprise various logics and data stores that, when executed, generate the medical session communication system server. Generally, a medical session communication server can act as the central hub, or data broker between patients and providers, while facilitating remote storage and access to patient virtual chart data. Additionally, the medical session communication server, as implemented within the private virtual cloud 135, may facilitate the matching, session establishment, and security of interactions between patients and providers.

In many embodiments, one of the ways the medical communication server may facilitate medical sessions between patients and providers is to acquire provider information data relating to a plurality of medical providers who may be willing to provide medical services via the medical session communication system 100. Such provider info data may be received within the memory 137 from a provider server 140 owned, operated, or otherwise authorized by a medical provider. In additional embodiments, the provider server 140 may be a dedicated machine within a doctor's office or home. In further embodiments, the provider server 140 can be a personal computer running one or more applications. In still further embodiments, the provider server 140 may be a portable computing device of the medical provider. As those skilled in the art can appreciate, the provider server 140 may comprise hardware that can be utilized by the medical session communication system 100 to facilitate sessions similar to client devices 110, and mobile computing devices 111, 112.

While a number of embodiments are described above with respect to FIG. 1, it should be recognized that a number of alternative embodiments may be utilized to practice a medical session communication system 100. By way of example and not limitation, the private virtual cloud 135 acting as a medical session communication server may instead be realized as a dedicated machine. Alternatively, a plurality of medical session communication servers may be utilized within the system 100. Similarly, the number of client devices 110 and provider servers 140 may scale with demand and is limited in number only by the available network resources available to facilitate such connections. An exemplary memory within a medical session communication server is discussed below.

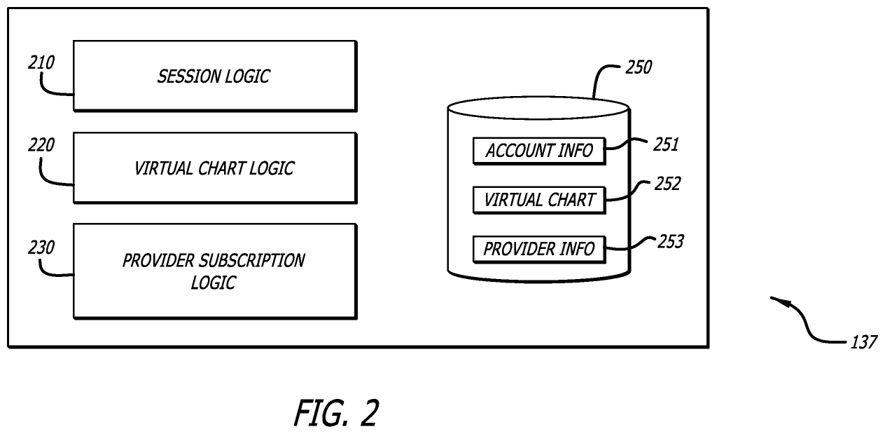

With reference to FIG. 2, an exemplary block diagram of a memory within medical session communication system in accordance with an embodiment of the invention is shown. A memory 137 residing within a medical session communication server (either via a private cloud computing service 135 or through a standalone device) is typically comprised of a number of logics 210, 220, 230 as well as a data store 250. While the logics and data stores depicted in FIG. 2 depict one embodiment, those skilled in the art will recognize that additional organizations can occur including embodiments with additional logics or multiple logics that are combined into a signle logic.

In many embodiments, a potential patient can download an application to facilitate a medical session. The medical session patient application may be provided to the patient via a code and/or instruction on how to download and execute the application. A new patient can then input their general personal info to create an account with the medical session communication system 100. In some embodiments, the personal information utilized during account creation is stored and utilized separately from virtual chart data. In other embodiments, the personal account data generated during the account setup process is subsequently utilized within the corresponding patient's virtual chart. In this way, a patient may be able to save time when inputting data.

In response to an account creation, the medical session communication system 100 can store the received account info 251 (i.e. patient account data) into the general data store 250. In a number of embodiments, in response to receiving new account info data 251, the virtual chart logic 220 may begin the process of generating a new virtual chart for the patient associated with the new account. In certain embodiments, a first step in the creation of a virtual chart is to generate and transmit a unique key code to the patient via their client device 110 or corresponding mobile computing device.

In certain embodiments, a patient may utilize a standalone client device 110 to sign up and create an account (perhaps for easy of data entry). In response to receiving the unique key code from the virtual chart logic 220, the patient may choose to continue the signup and virtual chart creation process on a mobile computing device or any other client devices 110 that may be hooked up or otherwise in communication with the patient's medical devices 115. It is contemplated that the unique key code may be sent directly to any device via electronic communication means as requested by the patient, including, but not limited to, texts, emails, etc.

Prior to the generation of a new virtual chart, a plurality of providers can be signed up to participate in medical sessions and have submitted their information into a provider info data (i.e. provider data) 253 within the data store 250. In many embodiments, the provider info 253 comprises a list of providers' names, locations, specialties, and/or availabilities. Those skilled in the art can recognize that provider info 253 can be periodically or aperiodically updated as new providers are added or removed from the service.

In a number of embodiments, the virtual chart logic 220 can generate a plurality of questions that can be transmitted to the patient to elicit responses. These questions can often include patient history questions, insurance information, and the types of services sought for medical sessions. In further embodiments, the virtual chart logic 220 can utilize provider info data 253 to generate questions specifically related to providers that have been subscribed to the medical session communication system 100. By way of example and not limitation, provider info data 253 can be used to generate questions regarding specific providers in response to patient answers regarding the types of services sought (e.g. a patient seeking skin care can be presented with available dermatologists), providers that accept the patient's insurance, and/or providers that are within a particular physical proximity of the patient as determined by a GPS signal.

Upon completion of the generated questions presented to the patient, any response data is transmitted back to the medical session communication server and is utilized by the virtual chart logic 220 to generate a completed virtual chart 252 associated with that particular patient. As those skilled in the art will understand, the virtual chart 252 can be stored in an encrypted format that satisfies current state, federal, or international laws and/or regulations involving the storage and access to confidential patient data. Virtually any method of encryption may suffice, including encryption methods that deploy a key and lock system placing the virtual chart data within the lock and providing the key to the patient via their client device 110 or mobile computing device, leaving the medical session communication server unable to access the data contained within the virtual chart unless accessed or in communication with the patient (and associated key).

When a patient attempts to establish a session with a medical provider, they may connect their client device 110 or mobile computing device (and associated medical session patient application) to the medical session communication server and request a provider. In a variety of embodiments, the provider subscription logic 230 can take a request for a session from a patient and generate an electronic communication to send to the selected provider to request a session. As those skilled in the art will recognize, the electronic communication can be any method that may be received and responded to by the medical provider. This may include, but is not limited to, texts, emails, push notifications, automated calls, and/or pages. The electronic communication can be configured to request a medical session with the medical provider. The medical sessions can comprise audio-only sessions, audio/video sessions, and/or virtual chart review (i.e. providing only medical data from the virtual chart to review by the medical provider). In many embodiments, the request for a medical session with a medical provider is transmitted in a secure/encrypted way. In certain embodiments, this can be accomplished through methods that encrypt a request at the medical session communication server such that the decryption data is only found within the medical provider server 140 or similar device.

In response to a medical provider accepting the request for a medical session with the patient, a request can be made from the provider server 140 to a session logic 210 that can establish a secure connection between the medical provider and the patient. In some embodiments, the secure connection is achieved as an audio/video session wherein the audio and video data streams are bi-directionally encrypted such that the provider server 140 encrypts the audio and video data of the medical provider and transmits to the client device 110 for encryption, and vice-versa. While any suitable encryption method can be utilized, the encryption can be facilitated by having the encryption/decryption methods operate on the client device 110 and provider server 140. However, in many embodiments, the session logic 210 can achieve a dual-step encryption method wherein the data is encrypted at either the client device 110 or provider server 140 and is then transmitted to the session logic 210 where it is decrypted, and then re-encrypted for transmission to the destination device where it is ultimately decrypted again for use. In this way, no encryption keys need to be exchanged between the patient and the medical provider.

Additionally, in response to a successful medical session being established, the virtual chart logic 220 can decrypt the virtual chart 252 and make it available to the medical provider for viewing and/or processing. During the medical session, the provider can have permissions established to add additional data to the virtual chart 252. Often, this can be notes that are entered during the medical session, or forms that have been scanned and can be appended to the virtual chart 252. In certain embodiments, the medical provider can only have access to amend the virtual chart 252 during the medical session. In further embodiments, the medical provider may have a pre-determined window of time to add data to the virtual chart after the session (in order to document the session and provide treatment plans, etc.) The pre-determined window may be facilitated by the patient providing the key to decrypt the virtual chart with a timed expiration (i.e. fuse). In still further embodiments, the medical provider may be able to provide additional virtual chart data after the medical session (or pre-determined window) by means of creating a second virtual chart file that may itself be encrypted and subsequently associated and stored with the original virtual chart 252 until such time that the patient accesses the virtual chart 252 again, thus decrypting the virtual chart 252 and allowing the virtual chart logic 220 to then merge the second virtual chart file into the original virtual chart 252, thus creating a single virtual chart 252.

When the medical session has ended, both the patient and the provider may be asked to respond to verify that the medical session termination was proper. In certain embodiments, the patient may receive an electronic communication configured to allow the user to respond in the affirmative or negative, and transmitting that response back to the session logic 210. Similarly, in some embodiments, the medical provider may receive a confirmation query to elicit a response regarding if the medical session was completed and that the patient should be assessed for the medical session. Upon confirmation by the patient and/or medical provider that the medical session was properly terminated, the session logic 210 can close out the secure connection and do any final “housework” or other “garbage collecting” processes required to uncouple the link between the patient, medical provider, and virtual chart 252. As discussed above, some of these processes may involve encryption methods.

Finally, the virtual chart logic 220 can be configured to provide constant access to a patient of their virtual chart 252. In this way, patients may be able to, for example, review notes made by the medical professional during the previous medical session, refreshing medical history recollection, and/or providing data to another, in-person medical provider. In a number of embodiments, provider info 253 may also be provided to the patient. Additionally, the patient may subsequently desire to adjust their account info 251, which can be allowed as long as the specific patient account info 251 is associated correctly with their virtual chart 252.

While a number of embodiments are described above with respect to FIG. 2, it should be recognized that a number of alternative embodiments may be utilized to practice a medical session communication system 100. By way of example and not limitation, the memory 137 may include greater or fewer logics and/or data types than depicted herein. Indeed, specific functions of logics described above may instead be executed by a different logic. It is contemplated that the memory 137 can reside in any suitable device that can establish a connection between a patient and medical provider. This may include dedicated servers, software applications, or combinations of the two. A flowchart describing an exemplary medical session is discussed below.

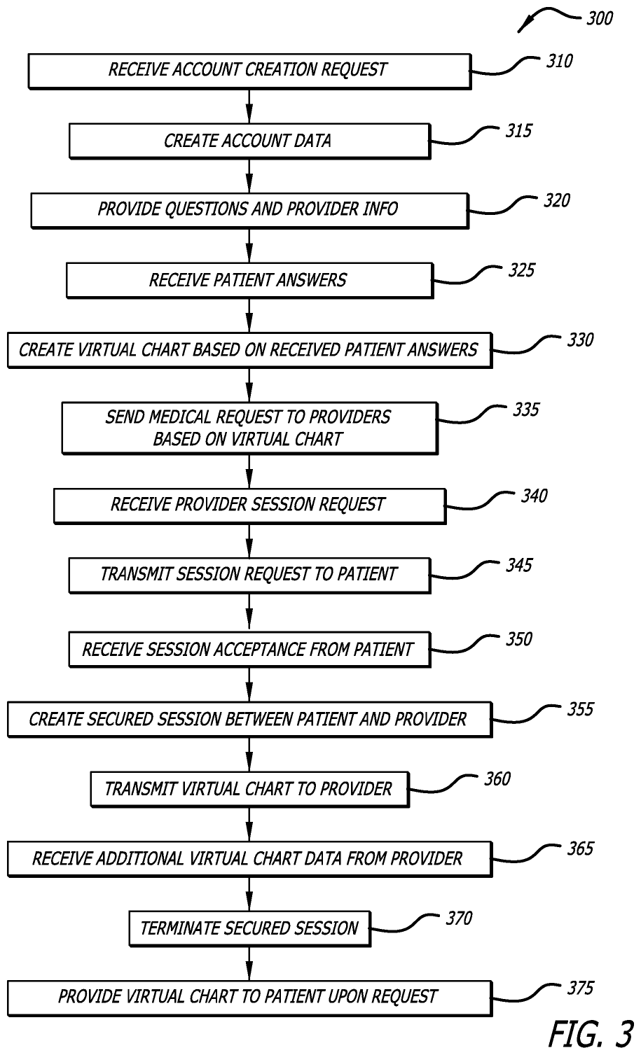

In regards to FIG. 3, an exemplary flowchart of a medical session communication process 300 in accordance with an embodiment of the invention is shown. The process 300 can often begin with the receiving of an account creation request (block 310). The account creation request is typically generated by a new patient to the medical session communication system 100. In response, account data is created (block 315) which may comprise general personal info including a log in name, password, and any other data necessary to create an account for the new patient within the medical session communication system 100. Once an account has been created, the process 300 provides a series of questions, often including provider info data to the patient (block 320). The patient can generate answers to the questions and give a selected provider (if needed) for their selected medical issue (block 325).

The process 300 can utilize the patient's submitted answers to generate a virtual chart (block 330). In response to any combination of the virtual chart, requested provider, or general health status questions answered, a medical provider can be sent a notification requesting a medical session with the patient (block 335). In response to the provider accepting the session notification request, a corresponding session request can be sent to the patient. In certain embodiments, the provider session request can be sent directly from the provider to the patient. However, in many embodiments, the medical session communication server may receive the session request from the provider first (block 340). In response, the medical session communication server can transmit the session request directly to the patient (block 345). The patient may then accept or deny the medical session request.

When the patient chooses to accept the medical session request, the acceptance response can be transmitted to and received by the medical session communication server (block 350). In response, session logic can begin to establish a secured connection between the patient and the provider (block 355). Upon successful creation of the secured connection, the virtual chart may be transmitted to the medical provider for examination (block 360). During the medical session, the provider can add in supplemental notes to the virtual chart which are then transmitted to and received by the medical session connection server (block 365). Eventually, the secured medical session will be terminated by the patient or medical provider (block 370). Subsequent to the medical session, the patient may be allowed to access and examine the virtual chart as desired (block 375).

While a number of embodiments are described above with respect to the process 300 described in FIG. 3, it should be recognized that a number of alternative embodiments and processes may be utilized to practice a medical session communication system 100. The exact ordering and inclusion of steps within the process 300 may be adjusted dependent on the needs of the system administrator, patient, or medical provider. By way of example and not limitation, the process 300 may include steps relating to encrypting and decrypting data. Further embodiments may include specific logics performing specific steps. Still further embodiments may include receiving and transmitting of data to and/or from medical devices to acquire and/or transmit data relating to specific health vectors.

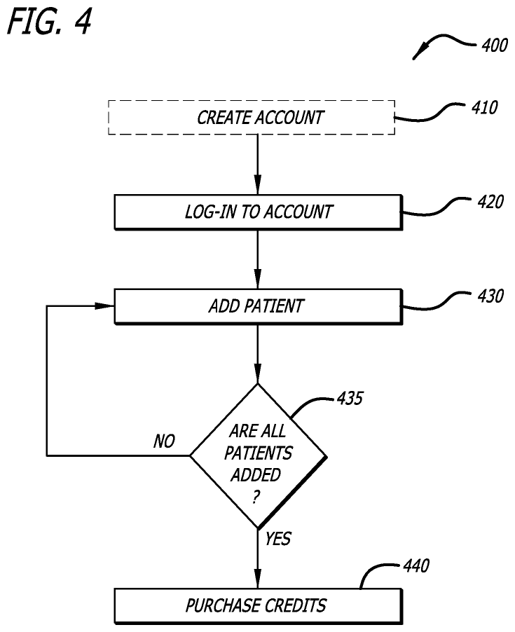

With reference to FIG. 4, an exemplary flowchart of a pre-session account setup process 400 in accordance with an embodiment of the invention is shown. In various embodiments, the medical session communication system can provide a means for patients to acquire care through one or more “credit” systems. In some embodiments, a patient can add a direct monetary amount within the system that can be utilized similar to a direct deposit of funds. In further embodiments, the patient can provide credits that are purchased for a flat fee that may be utilized for any particular service offered within the system.

The process 400 can begin for new users by creating an account within the medical session communication system (block 410). As discussed previously, account creation can utilize one or more publicly available log in systems to generate account data including, but not limited to, Facebook, Google, and/or LinkedIn sign-in services. In still further embodiments, the medical session communication system may be configured to work in tandem with, or as a separate client of a particular health insurance or provider system. In these embodiments, the patient may have a previously existing account with their provider and/or insurer which may then be imported or otherwise utilized to generate account data for the medical session communication system.

When an account has been created or is otherwise available, the process 400 may provide a means of logging into the account within the medical session communication system (block 420). As those skilled in the art will understand, logging in may comprise any standard method that can be utilized through either the chosen log-in service, or via device-specific log-in methods including, but not limited to, biometric identification (facial, fingerprint, etc.) motion, and/or traditional name/password pairing. As discussed above, externally generated accounts may be utilized which may require the medical session communication system to communicate with external servers and systems to provide, verify, and/or receive account data.

Prior to requesting a session, the patient must first add themselves or others to the medical session communication system (block 430). In certain embodiments, the patient info can be requested from an external source such as a medical provider and/or insurer. In further embodiments, the medical session communication system may generate a plurality of patient intake questions that are transmitted to the user for completion. Upon receiving verified patient intake data, the process 400 can successfully add the patient.

The medical session communication system can provide access to more than one patient within the system. Therefore, when a new patient is added, the process 400 can verify if all desired patients have been added to the system (block 435). If additional patients are desired to be added to the medical session communication system, the process 400 can again provide suitable methods for adding another patient (block 430). When all desired patients have been added the process 400 can then allow one or more patients to purchase credits or otherwise move forward with the session request process (block 440).

As discussed above, the purchasing of credits can be done within the app via one or more traditional methods of payment (credit card, e-check, external payment processor (PayPal, Amazon Pay, Google Pay, etc.), and/or Bitcoin or other blockchain-based transfer system). In a number of embodiments, credits may be purchased as a fixed block (e.g. 1 “credit” per $100) which may provide one or more benefits to providers within the system. For example, many providers prefer or require payment to be made prior to services rendered. In these cases, it may be difficult to provide a fixed price quote for a service as the initial issue that a session is meant to address changes to another issue upon further diagnostic questions or interaction by the provider. The continued fluctuation in potential price can sometimes leave providers giving more service than was otherwise paid for. Utilizing a fixed block credit system, providers may more easily set rates for services (e.g., symptom A requires 1 credit per session, while symptom B requires 2 credits per session, etc.). Additionally, fixed block credits may also help to provide means of issuing sales or transferring credits between patients without the need to associate said transfers with cash values. Lastly, in some jurisdictions, fixed block credits can be used in lieu of stored cash values to avoid undesired regulations or accounting rules that are typically attached to those stored cash values.

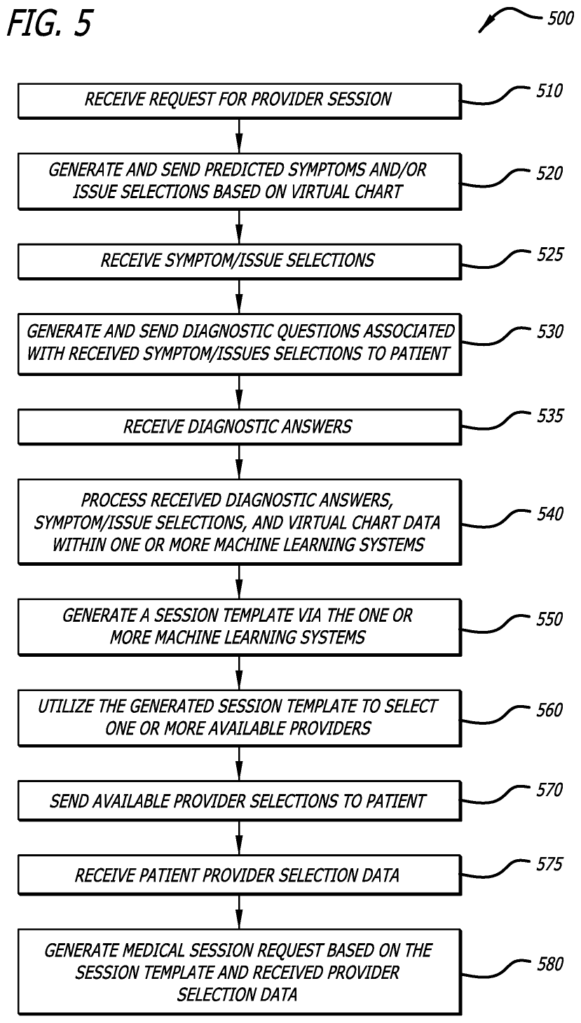

With reference to FIG. 5, an exemplary flowchart of session template generation in accordance with an embodiment of the invention is shown. In many embodiments, the medical session communication system can utilize one or more machine learning processes to generate a session template that can guide the medical providers during the connected session. The session template may, in various embodiments, include an initial diagnosis that can be utilized or verified by the medical provider. In various embodiments, the process 500 of generating these session templates supplements the process described in the discussion of FIG. 3.

The process 500 for achieving this typically includes receiving a request for a provider session (block 510). This request can be generated within a patient device that has the related medical session communication system application. When the request has been received, the process 500 can generate and send predicted symptoms and/or issues that the patient may request which is determined based on the previously stored and/or available virtual chart (block 520).

For example, the patient may have data within their virtual chart that indicates a history of diabetes. The medical session communication system may, for this patient, provide a list of potential issues related to diabetes as an initial selection screen. While the patient may require a non-diabetes related session, providing predicted symptoms and/or issues can reduce the time needed from the patient to continue the session request process. The patient, via their medical session communication system application or other software, can respond with selections associated with the current symptom/issue that they are experiencing, which is received by the medical session communication system (block 525).

Once the medical session communication system has a plurality of symptom and/or issue selection data, the process 500 can follow-up by generating and sending one or more diagnostic questions associated with the received selection data (block 530). For example, if the patient had indicated that their current issue was a prolonged headache, the generated diagnostic questions may include asking how long the current headache has persisted, how often does the patient get similar headaches, provide a rating to indicate the current strength of the headache or associated pain, etc. These answers to the diagnostic questions can be sent back by the patient via the medical session communication system application and be received by the system (block 535).

Once the medical session communication system has symptom/issue selection data, diagnostic answer data, and virtual chart data associated with the same patient, the process 500 process this data as an input into one or more machine learning systems (block 540). In certain embodiments, the processing of this patient data can occur to generate a particular input within a machine learning system. In additional embodiments, the processing may be necessary to strip or otherwise obfuscate identifying patient data that may be subject to the Health Insurance Portability and Accountability Act (HIPAA) or other similar patient data privacy laws within various jurisdictions. In still additional embodiments, the processing of the data may further include formatting for transmission to an external or third-party machine learning system.

When processed by one or more machine learning systems, the process 500 may be configured to generate a session template and/or session template data that can be utilized in other components, systems, or devices to generate a complete session template (block 550). A session template can be utilized by the medical provider to guide a particular session based on all available data and initial diagnostics available via the machine learning systems. By way of example and not limitation, a session template may be configured to provide a list of questions that can be optional or required for a provider to answer and fill out during a session in order for a session to be considered completed and able to be closed and processed for payment. If a patient presented with a shortness of breath in the chest, the session template can be configured to verify that the medical provider has asked questions related to further stroke and/or potential heart attack symptoms. In this way, evidence to avoid potential future liability can be generated as well verifying that the standard level of care was provided to the patient.

Once a session template is available, the process 500 can utilize the session template to select one or more available providers (block 560). As can be appreciated by those skilled in the art, various medical providers can have different specialties such that they may be best suited or at least competent to work with and diagnose a patient in one area of care, while being unable to provide the same (or standard) level of service in another area of care. Thus, session templates, or the data and/or questions contained within may be used to screen for providers that may best provide care for the patient during this particular session request. For example, in response to a patient having an issue with their foot, a session template may be generated that requires questions and/or remote visual examinations of the patient's foot and related history. A provider who specializes in podiatry will typically be better suited to provide this patient sufficient care compared to an otolaryngologist who specializes in ears, noses, and throats.

Based on available resources, user and/or provider preferences, or other restrictions, the medical session communication system can filter out providers which can then be sent to the patient for selection (block 570). The provider selection data may include, but is not limited to, provider names, available times, pictures, specialties, ratings, reviews, and/or previous interactions with the patient if available. The patient can select a provider from this selection and transmit the associated provider selection data which is received by the medical session communication system (block 575).

Once the patient provider selection data is received, a medical session request may be generated by the medical session communication system which will be sent to the medical provider for approval (block 580). Generation of the medical session request may utilize both patient provider selection data and session template data. In this way, the request sent for approval to the medical provider may include not just the general request and associated patient information but may also include the session template data which may be formatted to show the potential medical provider what the potential session may look like. This may further inform the provider whether they will accept or reject the session request.

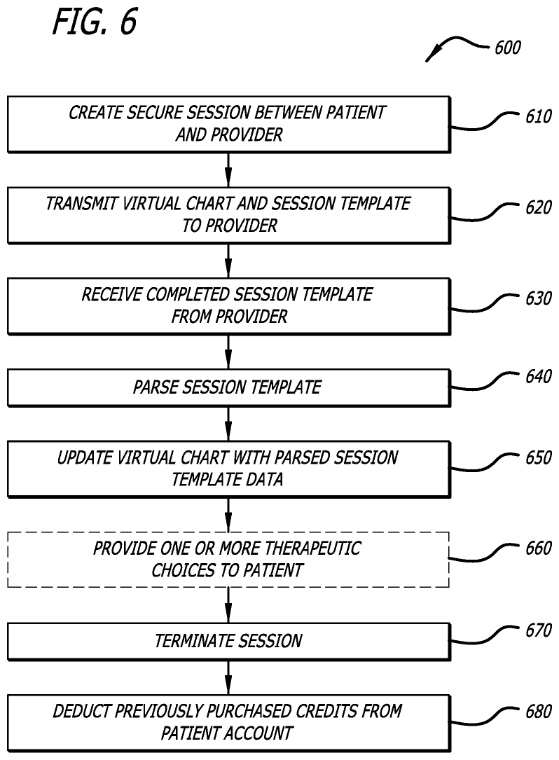

With reference to FIG. 6, an exemplary flowchart of a secure session interaction in accordance with an embodiment of the invention is shown. The process 600 may be utilized to supplement or otherwise supplant the relevant portions of the process 300 described in FIG. 3. The process 600 generally describes a particular method of establishing, terminating, and cashing out a secure medical session within the medical session communication system.

Once a session request has been approved by a medical provider, the process 600 can create a secure session between the patient and the provider (block 610). In certain embodiments, the creation of the secure session may begin immediately after the acceptance of the session request by the medical provider. In other embodiments, sessions may be scheduled for a future pre-determined time window which can then be created upon arrival by one or more parties within the session. In additional embodiments, the creation of the secure session may be between multiple patients and/or providers. This may occur when, for example, a second opinion is requested or when the medical provider wishes to bring in another provider to consult or take over the session in response to changes in the potential diagnoses during the session.

Once established, the process 600 can transmit the patient's virtual chart (or related data) and session template (or related data) to the provider (block 620). In some embodiments, the session template data is sent which is then formatted to generate the session template on the medical provider's device. In further embodiments, the medical provider has a companion application associated with the medical session communication system that receives and process the session template data to generate the session template. In certain embodiments, a web-based portal or application can be utilized that transmits a fully processed session template to the provider for use.

Once received, the medical provider can facilitate the secure session with the patient(s) which can allow for the completion of the session template. As the secure session completes, the session template should also be completed by the medical provider. In some embodiments, the session template may include fields that can be completed by the medical provider after communication with the patient has concluded. Once the provider has completed the session template, it may be transmitted to and received by the medical session communication system (block 630).

A completed session template can be parsed by the medical session communication system (block 640). Parsing can include, but is not limited to, extracting responses provided by the medical provider, cross-referencing answers with one or more machine learning systems for verification or further follow-up activity, and/or determining what (if any) therapeutic choices are to be provided to the patient. Data parsed from the session template can be utilized to update the patient's virtual chart (block 650).

When a medical provider has determined that the patient would benefit from one or more therapeutic methods, these choices can be presented to the patient (block 660). For example, in response to the secure session between the patient and medical provider, the provider may determine that a prescription medication is required. The patient may then be presented with various choices of (for example) which location the patient would like to pick up the prescription, what insurance (if any) may pay or require, and if they prefer a generic option if available. These choices can further be completed outside of the secure session in particular embodiments.

Once communication between the patient and medical provider and the medical session communication system has stopped, the session can be terminated (block 670). Termination can be dictated by a particular set of requirements within the medical session communication system. For example, a session may not terminate and continue in the process 600 unless the medical provider has answered each question within the session template. In additional embodiments, the session can fail to terminate until both parties (patient and provider) agree to end the session. In still additional embodiments, the disconnection of communication between patient and provider may not be classified as a termination of the session and instead provides means to reestablish the connection.

Once the session has been successfully terminated, the process 600 can begin to deduct the previously purchased credits (or stored cash value) from the patient account (block 680). As discussed above, the patient can purchase credits or add value to their account which can be used or provided for service by the medical provider. Deduction of those funds can be configured to only occur once the session has fully terminated. In further embodiments, other requirements may be configured within the medical session communication system to dictate when the deduction may occur.

While the invention has been described in terms of particular variations and illustrative figures, those of ordinary skill in the art will recognize that the invention is not limited to the explicit variations and/or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. To the extent there are variations of the invention, which are within the spirit of the disclosure or equivalent to the inventions found in the claims, it is the intent that this patent will cover those variations as well. Therefore, the present disclosure is to be understood as not limited by the specific embodiments described herein, but only by scope of the appended claims.

What is claimed is:

1. A method of providing computerized medical communications comprising:
receiving patient account data associated with a patient;
generating a plurality of questions configured to elicit responses from the patient;
transmitting the generated plurality of questions to the patient;
receiving response data from the patient wherein the response data is generated based on the transmitted plurality of questions;
generating virtual chart data with a virtual chart logic based on the received response data;
receiving provider data with a provider subscription logic comprising subscribed providers; and
generating a secure session with a session logic between the patient and a provider based on at least the patient account data and the provider data;
providing the virtual chart data to the provider during the secured session;
terminating the secured session; and
providing a method of acceptance to the patient to indicate an approval of the secured session termination.

2. The computerized medical communications method of claim 1, wherein the patient account data is received from an external application.

3. The computerized medical communications method of claim 2, wherein the external application is executed on the patient's mobile computing device.

4. The computerized medical communications method of claim 1, wherein the generating of the plurality of questions further includes retrieving provider data and generating a plurality questions relating to providers based on the retrieved provider data.

5. The computerized medical communications method of claim 1, wherein the receiving of response data from the patient further includes receiving a response associated with a provider selected by the patient to provide medical care.

6. The computerized medical communications method of claim 5, wherein the method, in response to receiving a provider selection response, further generates an electronic communication to the provider configured to elicit the generation of a secured session between the provider and the patient.

7. The computerized medical communications method of claim 1, wherein the generated virtual chart data comprises health data provided by the user.

8. The computerized medical communications method of claim 7, wherein the health data provided by the user is obtained from the patient's mobile computing device.

9. The computerized medical communications method of claim 1, wherein the method, upon receiving an acceptance of the secure session termination, adds data acquired during the secure session to the virtual chart data.

10. The computerized medical communications method of claim 9, wherein the method further comprises providing patient access to the data added after the termination of the secure session.

11. A computerized medical communication system comprising:
a processor; and
a memory communicatively coupled to the processor, the memory comprises:
a virtual chart logic configured to:
receive patient account data relating to a patient;
generate a plurality of questions configured to elicit responses from the patient;
generate virtual chart data based on answers received from the patient in response to the generated plurality of questions;
provide the virtual chart data within an authenticated session;
a provider subscription logic configured to receive provider data in response to medical providers subscribing to the medical communication system; and
a session logic configured to:
generate a secure session between the patient and the provider based on at least the patient account data and the provider data; and
provide, in response to the termination of the secure session, a method of acceptance to the patient to indicate an approval of the secure session termination.

12. The computerized medical communication system of claim 11, wherein the plurality of questions are further configured for determining one or more symptoms from the patient.

13. The computerized medical communication system of claim 11, wherein the session logic is further configured to generate a session template.

14. The computerized medical communication system of claim 12, wherein the session template is generated utilizing one or more machine learning systems.

15. The computerized medical communication system of claim 13, wherein one or more of the machine learning systems is a third-party system.

16. The computerized medical communication system of claim 13, wherein the one or more machine learning systems utilize the virtual chart data to generate the session template.

17. The computerized medical communication system of claim 11, wherein the generated plurality of questions comprises questions relating to providers subscribed to the computerized medical communication system.

18. The computerized medical communication system of claim 11, wherein the memory further comprises account data associated with the patient.

19. The computerized medical communication system of claim 18, wherein the account data comprises at least patient credit data.

20. The computerized medical communication system of claim 19, wherein the patient credit data can be deducted by the session logic upon successful termination of a secure session.
